(19) United States
(12) Patent Application Publication
(10) Pub. No.: US 2023/0142838 A1
(43) Pub. Date: May 11, 2023

(54) DEVICES AND METHODS FOR NUCLEIC ACID EXTRACTION-FREE STI PATHOGEN TESTING

(71) Applicant: SUMMIT BIOLABS, INC., Aurora, CO (US)

(72) Inventors: Robert E. Blomquist, Boulder, CO (US); Shi-Long Lu, Englewood, CO (US)

(21) Appl. No.: 17/972,111

(22) Filed: Oct. 24, 2022

Related U.S. Application Data
(60) Provisional application No. 63/277,061, filed on Nov. 8, 2021.

(51) Int. Cl. C12Q 1/686

(52) U.S. Cl. CPC C12Q 1/686; G01N 2800/26

(57) ABSTRACT

The invention provides compositions, devices, methods and kits allowing for rapid diagnosis of diseases and pathogens, including sexually transmitted infectious diseases, via nucleic acid extraction-free, direct PCR techniques.

### TECHNICAL FIELD

The invention generally relates to diagnostic methods, and, more particularly, to compositions and methods for performing extraction-free pathogen testing and detection, especially for sexually transmitted infections.

### BACKGROUND

The United States Center for Disease Control estimates that 1 in 5 individuals have a sexually transmitted infection (STI) and that every year there are over 25 million new STI cases. Consequently, in the US, an estimated $16 billion is spent annually on direct medical costs attributed to STIs. These costs are likely to increase as antibiotic-resistant strains of bacterial STIs become more prevalent and other STIs emerge, such as with Monkeypox in 2022.

STIs are attributable to myriad viruses, bacterium, and microorganisms. The most common STI is Chlamydia trachomatis (CT) infection, with over 1.5 million new cases in the United States annually. Although about half as prevalent, Neisseria gonorrhoeae (NG) infection is becoming an increasing concern with the emergence of strains resistant to ceftriaxone, the first-line treatment for NG infection.

Whether an STI is treatable or not, early detection and surveillance remains a key component in reducing the societal burden and spread of STIs.

Current detection techniques for many infectious diseases involve the use of polymerase chain reaction (PCR). PCR is a technique used to selectively amplify a specific region of DNA of interest (the DNA target). For example, various real-time PCR assays (also referred to as quantitative PCR (qPCR)) for detecting many viral and bacterial infections have been developed worldwide.

However, while current PCR methods allow for the detection and diagnosis of infectious diseases, those methods suffer from drawbacks. One notable drawback is that current approaches rely on an initial step of isolating and purifying nucleic acids from a clinical sample as part of the testing protocol. The initial nucleic acid isolation and purification step (i.e., extraction step) required in conventional methods, prior to undergoing PCR, constitutes a major bottleneck in the diagnostic process, as it remains both manually laborious and expensive, and further increases the chances of accidental contamination and human error.

Furthermore, the efficacy of PCR-based tests for diagnosing infections, in particular sexually transmitted infections, varies based on the type of sample analyzed (e.g., genital swab, urine, blood, or saliva), the timing of sample collection relative to the course of an infection, and even the behavior of subjects prior to sample collection.

### SUMMARY

The present invention provides compositions and methods for rapid, extraction-free detection and analysis of nucleic acid in a biological sample. More specifically, the invention provides compositions for processing a biological sample and providing usable nucleic acid for subsequent amplification and/or detection (for example, using next generation sequencing technologies), while eliminating the need for an initial nucleic acid extraction step. Moreover, compositions of the invention eliminate the need for any pathogen transport media, which are known to inhibit subsequent PCR assays. Compositions of the present invention include, for example, a unique buffer composition for sample transport and preparation that, when mixed with a sample of interest, is capable of stabilizing and preparing nucleic acid from the sample for direct amplification and analysis, without the need for initial nucleic acid extraction (i.e., isolation and purification of the nucleic acid).

Advantageously, the methods of the invention can use non-invasive sample types, such as swabs taken from a potentially infected area. Buffers used in the extraction-free methods of the invention stabilize and preserve target nucleic acids (e.g., from one or more sexually transmitted pathogens) from the non-invasive samples. This allows samples to be taken at home, sent to a lab for analysis or even analyzed at home on a suitable point-of-care testing device. This can be critical to user adoption of a testing modality, particularly when testing for sexually transmitted infections (STIs). Self-collection of samples for STIs has been shown, especially in men, to be readily accepted. See, Yared N et al., Optimizing Screening for Sexually Transmitted Infections in Men Using Self-Collected Swabs: A Systematic Review. Sex Transm Dis. 2018 May; 45(5):294-300, which is incorporated herein by reference.

In the methods of the invention, sample testing is direct from sample without nucleic acid extraction steps. Instead, after clinical samples are provided with the unique buffer composition described herein, nucleic acids from the samples may be used directly in downstream assays, including qPCR, rtPCR, and/or NGS-based diagnostic testing. The invention is useful for the detection of DNA or RNA, as required, for detection of one or more sexually transmitted pathogen. Accordingly, in preferred aspects, target nucleic acids for detection include nucleic acid sequences associated with one or more sexually transmitted pathogens.

Methods of the invention are applicable to the detection of any pathogen that is amenable to PCR amplification and includes viruses (such as human papillomavirus (HPV) and monkeypox virus (MPV)), bacteria (such as Chlamydia trachomatis (CT) and Neisseria gonorrhoeae (NG)), and other pathogens (e.g., Candida albicans yeast). In certain aspects, methods of the invention may detect a plurality of sexually transmitted pathogens from a single sample, which may include a combined sample. Further, methods of the invention are amenable to detecting oncogenic viruses, some of which are sexually transmitted (such as HPV). By extension, the methods of the invention may detect one or more cancers (such as lung, head and neck, cervical).

Accordingly, in certain aspects, methods of the invention may include detecting one or more genetic markers correlated with an elevated risk of cancer. Such genetic markers may be those correlated with a particular pathogen or pathogen variant, for example, those used to discriminate high-risk variants of HPV, including HPV-6, HPV-11, HPV-16, HPV-18, HPV-31, HPV-33, HPV-35, HPV-39, HPV-45, HPV-51, HPV-52, or HPV-68 (See, American Cancer Society, Human Papilloma Virus (HPV), Cancer, HPV Testing, and HPV Vaccines: Frequently Asked Questions (Oct. 22, 2013). Similarly, genetic markers correlated with an elevated risk of cancer may include oncogene sequences and/or a gene mutation sequence (such as KRAS G12C-mutated NSCLC). Exemplary genetic markers include, for example, those associated cervical cancer, such as SC6; SIX1; human cervical cancer 2 protooncogene (HCCR-2); p27; virus oncogene E6; virus oncogene E7; p16INK4A; Mcm proteins (such as Mcm5); Cdc proteins; topoisomerase 2 alpha; PCNA; Ki-67; Cyclin E; p-53; PAIl; DAP-kinase; ESR1; APC; TIMP-3; RAR-β; CALCA; TSLC1; TIMP-2; DcR1; CUDR; DcR2; BRCA1; p15; MSH2; RassflA; MLH1; MGMT; SOX1; PAX1; LMX1A; NKX6-1; WT1; ONECUTI; SPAG9; and Rb (retinoblastoma) proteins.

Preferred methods of the invention are used to detect one or more sexually transmitted infections (STIs) from a minimally-invasive sample obtained from a subject. For example, in preferred aspects, a sample used in methods of the invention is obtained as a mucosal membrane swab. Such samples may include one or more of a vaginal swab, a cervical swab, a urethral swab, a genital swab, a buccal swab, a throat swab, a nasal swab, ocular swab, and a combination of any thereof. In certain aspects, methods of the invention use a fluid sample from a subject, which may include one or more of urine, vaginal mucosa, saliva, blood, nasal mucosa, sputum, cerebrospinal fluid, pus, breast nipple aspirate, ascites, lymphatic fluid, sweat, lacrimal fluid, and a combination of any thereof. In certain aspects a combined swab and fluid sample is used in methods of the invention to detect sexually transmitted diseases.

Preferably, samples include one or more non-invasive mucosal membrane swabs and/or fluid sample (e.g., urine and/or saliva). Non-invasive sampling allows for patients to collect samples in their own homes or remote clinics without on-site access to sophisticated laboratory equipment and staff. Advantageously, the extraction-free methods of the invention use proprietary buffer compositions, which allow target nucleic acids from the samples to be preserved and secured for shipping to a laboratory for analysis. Fortuitously, the extraction-free methods of the invention and use of the proprietary buffer compositions also allows for target nucleic acids from the sample to be analyzed at home on an appropriate point-of-care testing device.

As methods of the invention enable sample collection for STI testing at home, the methods provide several advantages over traditional in-clinic testing, including privacy. Thus, methods of the invention may help those, who due to a perceived stigma, are reluctant to seek in-person testing. Moreover, even when not provided in-home, methods of the invention may be used in fairly austere locations, and the samples collected by minimally-trained staff. This finds distinct utility in expanding STI testing beyond centralized locations, e.g., hospitals with specialized staff, to underserved communities.

In one aspect, the invention allows the combination of two or more different sample type in a single assay, thus allowing more accurate results, especially when testing for multiple sexually transmitted pathogens, which may present. In certain aspects the two or more different sample types comprise two or more different types of mucosal membrane swab. In certain aspects the two or more different sample types comprise two or more different types of fluid sample. In certain aspects, the two or more different sample type comprise one or more mucosal membrane swab (e.g., a vaginal swab) and one or more fluid sample (e.g., saliva or urine). Different STIs, including as differentially presenting across a population, may provide target nucleic acids of varying quality and/or quantity depending on the type of sample. For example, patients may have multiple STIs, each only detectable in a particular location on the subject's body (e.g., a CT infection reliably detected from a genital swab and a latent HPV infection detected from a buccal swab). Using methods of the invention, the different sample may be combined in the buffers disclosed herein for transport to a lab for further analysis.

Methods of the invention may be used to detect any sexually transmitted infection. Exemplary STIs detected by methods of the invention include bacterial vaginosis, CT, cystitis, NG, hepatitis A, hepatitis B, hepatitis C, herpes (herpes simplex type 1 and 2), HIV, HPV, MPV, lymphogranuloma venereum, molluscum contagiosum, non-gonococcal urethritis, pelvic inflammatory disease, phthirus pubis, syphilis, trichomoniasis, and vaginitis. In certain aspects, methods of the invention detect a plurality of sexually transmitted infections from a single sample. In preferred aspects, methods of the invention detect CT and/or NG infection in a sample. Methods of the invention may be used to detect oncogenic viruses. Exemplary oncogenic viruses detected by methods of the invention include HPV, Epstein-Barr virus (EBV), hepatitis C and virus (HCV).

In another aspect, the invention provides a stabilizing buffer that preserves the nucleic acids of one or more sexually transmitted pathogens in a sample. The buffer, described below, stabilizes the nucleic acids of viruses, bacteria, and other pathogens for transport prior to detection of pathogenic nucleic acids. In a preferred embodiment, a transport buffer as described herein is added to a liquid sample suspected of containing a pathogen. The sample is then transported to a laboratory for extraction and testing. Because buffer compositions disclosed herein preserve target nucleic acids, multiple pathogen detection assays can be run in a single sample and/or sample types can be combined for multiplex pathogen analysis.

In a first aspect, the invention provides compositions for processing samples, including combined samples, as described herein, and providing usable nucleic acid for subsequent amplification and/or detection (for example, using next generation sequencing technologies), while eliminating the need for an initial nucleic acid extraction step. Compositions of the invention eliminate the need for pathogen transport media, which typically inhibit PCR. Compositions of the present invention include, for example, a unique buffer for sample transport and preparation that, when mixed with a sample of interest, is capable of preparing nucleic acid from the sample that is suitable for direct nucleic acid amplification and analysis without the need for initial nucleic acid extraction (i.e., isolation and purification of the nucleic acid).

In certain aspects, the present invention includes kits with all the necessary components to obtain a combined sample, which may preferably be one or more mucosal membrane swabs or a urine sample. This may include providing patients with a kit. The subject can use the simple-to-use components of the kit, in the comfort of their home, to provide a sample. Using the proprietary buffer compositions disclosed herein, the sample can be adequately preserved and secured, such that it can be mailed to a laboratory for analysis.

For purposes of the invention, a target nucleic acid may be a human genomic sequence, a human transcript sequence, an oncogene sequence, a gene mutation sequence (such as KRAS G12C-mutated NSCLC), a pathogen sequence or a parasitic sequence.

Preferred methods further include mixing a sample with an inventive buffer composition that is capable of preparing nucleic acid from the biological sample suitable for nucleic acid amplification without initial extraction of the nucleic acid. In other words, upon mixing of the biological sample with the buffer, specific components within the buffer allow for nucleic acid from the sample to be sufficiently prepared for subsequent nucleic acid analysis (i.e., amplification via PCR) without requiring the typical extraction (isolation and purification) step.

Buffer compositions used in the methods of the invention generally include nuclease-free water, an antifungal solution, an antibiotic solution, a ribonuclease inhibitor, a reducing agent solution and/or a Tris-Borate-EDTA buffer solution. In certain aspects, the buffer composition also serves as a transport medium, in a sample, including any sample collection swab(s) is immediately placed within an appropriate collection vessel containing the buffer composition.

Methods further include performing one or more PCR assays on the prepared nucleic acids to detect one or more target pathogenic nucleic acids. Upon detection of a target nucleic acid, a patient may be diagnosed as having an STI.

The step of performing PCR assays includes using target nucleic acid specific primer-probe sets. In certain methods, the target nucleic acid specific primer-probes are specific for target nucleic acids of different pathogens in a single sample. In some embodiments, the step of performing the PCR assay includes using a primer-probe set specific to ribonuclease P (RNP). Extraction methods disclosed herein are also useful for detecting human genomic or RNA sequences, as methods are agnostic as to the source of nucleic acid.

In certain aspects, methods of the invention further include quantifying a pathogenic nucleic acid. For example, performing the one or more PCR assays includes performing at least one of quantitative PCR (qPCR) and digital PCR (dPCR), which may include droplet digital PCR (ddPCR). In addition to diagnosing the patient as either having been or not been infected with a sexually transmitted pathogen, the method may further include the step of determining the severity of the infection based on the pathogenic nucleic acid quantity. In some embodiments, methods may further include the step of comparing pathogenic nucleic acid quantities in a plurality of biological samples obtained from the patient at successive time points and determining disease progression based on increases or decreases in the nucleic acid quantities over time. Methods of the invention may further include predicting disease outcomes based on the identity or quantity of target nucleic acid in a sample. Methods of the invention may also be used to inform a course of treatment or prognosis. For example, results can be used to determine an appropriate therapeutic or clinical procedure.

In another aspect, the invention provides for detection of bacteria using extraction-free buffer to preserve bacterial DNA and/or RNA for detection. The same buffer is useful for preservation of both virus and bacteria, thus allowing detection of viral and bacterial pathogens in the same sample or combination of samples. Thus, in one aspect the invention provides methods of stabilizing bacteria and/or virus in a biological sample for extraction-free testing via, for example, PCR. The invention therefore allows simultaneous detection of viral and bacterial samples. This allows for an “all-in-one” test for viral and bacterial sexually-transmitted infections (STI), such as Chlamydia trachomatis and Neisseria gonorrhea. In addition, because the buffers disclosed herein stabilize viruses as well, a single test and sample may also be used to detect viral STIs, such as HPV, HIV, MPV, and herpes.

The present invention also provides methods for extraction-free analysis of nucleic acid. An exemplary method includes mucosal membrane swab sample from a subject. Alternatively or additionally, the method may include obtaining an additional mucosal membrane swab (e.g., from a another bodily location) and/or one or more fluid samples (e.g., urine). Where a plurality of samples is used, methods of the invention may include combining the samples in a vial. Samples (including combined samples) are mixed in the vial with a preservation buffer composition, which includes, for example buffer nuclease-free water, an antifungal, an antibiotic, and a ribonuclease inhibitor. Thus, methods include directly amplifying nucleic acid in the buffer with primers specific to a target nucleic acid. Direct amplification occurs without a prior nucleic acid extraction step. After amplification, the method includes analyzing amplicons produced in said amplifying step to detect presence of one or more pathogen.

In certain aspects, the sample is a fluid sample. Fluid samples may be obtained using a collection aid. For example, if the fluid sample is saliva, the sample may be obtained from a subject using a sample collection aid or a funnel. The sample collection said may include the buffer composition, which is released into the vial. For example, the sample collection aid may include the buffer composition in an internal pouch or compartment or in the lid, which releases the buffer composition into the vial. In certain aspects, the sample collection aid or funnel includes a lid. The lid may include the buffer composition, which is released into the vial when the lid is closed. In certain aspects, the sample collection aid or funnel is integrated with a vial. Alternatively, the sample collection aid or funnel may be configured to couple to the vial during saliva collection. In certain aspects, the sample collection aid or funnel is configured such that it can be reversibly coupled to the vial.

In preferred aspects, the sample is obtained using one or more mucosal membrane swabs. In certain aspects, the mucosal membrane swabs include one or more of a vaginal swab, a cervical swab, a urethral swab, a genital swab, a buccal swab, a throat swab, a nasal swab, ocular swab, and a combination of any thereof. In certain aspects, a swab used to obtain mucosal membrane sample is attached to a cap used to seal the vial. Sealing the vial with the cap may place the swab in a fluid sample to form a combined sample. Alternatively, a first swab may be added to a vial and a second swab is added. One of the swabs may be attached to the vial cap.

The present invention also provides kits for performing the methods of quantifying the nucleic acids, including viral and/or bacterial nucleic acids, as disclosed herein. In certain aspects, a kit of the invention includes one or more vials, sample collection aid and/or funnel; a buffer composition, such as a transport (preservation) buffer, primers for amplifying one or more target nucleic acid, and instructions for use.

### BRIEF DESCRIPTION OF THE DRAWINGS

FIG. 1 shows a schematic overview of an extraction-free, real-time RT-qPCR test intended for the qualitative detection of nucleic acid from one or more sexually transmitted pathogens in mucosal membrane swabs collected and processed via unique buffer compositions of the present invention.

FIG. 2 shows a sample from a patient suspected of having an STI and loading of the sample into an instrument capable of performing one or more assays on the sample to determine whether viral nucleic acid associated with the viral infection is present.

FIG. 3 shows results for a SARS-CoV-2 qPCR detection protocol performed on paired saliva-only and combined saliva and nasal swab samples obtained from the same patients.

FIG. 4 shows select components used in methods of the disclosure and provided in certain kits of the invention.

FIG. 5 shows select components of a kit of the invention for detecting a target nucleic acid in a combined sample.

FIG. 6 shows qPCR readouts for assays using the nucleic acid extraction free methods of the invention to detect CT and NG nucleic acids from swab and fluid samples.

FIG. 7 summarizes the qPCR assay of FIG. 6 and its resulting data for singleplex assays using probes and primers for NG.

FIG. 8 summarizes the qPCR assay of FIG. 6 and its resulting data for singleplex assays using probes and primers for CT.

FIG. 9 shows qPCR readouts for assays using the nucleic acid extraction free methods of the invention to detect CT and NG nucleic acids from swab and fluid samples.

FIG. 10 summarizes the qPCR assay of FIG. 9 and its resulting data for singleplex assays using probes and primers for NG.

FIG. 11 summarizes the qPCR assay of FIG. 9 and its resulting data for singleplex assays using probes and primers for CT.

FIG. 12 summarizes the qPCR assay of FIG. 9 and its resulting data for multiplex assays using probes and primers for CT and NG.

FIG. 13 provides a chart showing the consistent results across samples and assays using the nucleic acid extraction-free methods of the invention for detecting STIs.

### DETAILED DESCRIPTION

The present invention provides compositions, methods, and kits allowing for rapid diagnosis of sexually transmitted diseases via extraction-free, direct PCR techniques using minimally-invasive samples. The invention also provides a buffer that stabilizes and preserves target nucleic acids in a sample and allows extraction-free testing of pathogen nucleic acid and, in particular, multiple pathogens simultaneously from one or more sources (e.g., multiple samples from an individual). Thus, methods of the invention include methods for viral testing, bacterial testing, or combinations. Moreover, because buffers taught herein preserve samples, such as nucleic acids from viral STIs (e.g., HPV and HIV), bacterial STIs (e.g., CT and NG), other pathogens, and oncogenes, the samples can be transported without substantial loss of the target pathogen and/or nucleic acid sequence.

Compositions, methods, and kits of the invention may be used for processing a biological sample and providing usable DNA for subsequent PCR assays, while eliminating the need for an initial nucleic acid extraction step. The present invention includes a unique buffer composition for sample transport and preparation that, when mixed with a sample of interest, is capable of preparing nucleic acid from the sample that is capable of being directly used for nucleic acid amplification and analysis without the need for initial nucleic acid extraction (i.e., isolation and purification of the nucleic acid). Accordingly, unlike many prior approaches, which include a nucleic acid extraction step, the direct sample testing of the present invention simplifies this process by omitting the extraction step. Instead, after clinical samples are provided in the unique buffer composition, a pathogen may be inactivated either through heating or by direct lysis in the buffer. The inactivated samples can then be used for downstream qPCR diagnostic testing.

As a result, compositions, methods, and kits of the present invention improve upon conventional pathogen testing and detection approaches by reducing the number of steps required for sample preparation and testing. In turn, the time required for testing is greatly reduced, resulting in faster turnaround times and delivery of results. Furthermore, the present invention reduces the cost of labor and consumables, while further reducing cross contamination of samples as well as infections of the samples to operators. The efficiency and costs saving are magnified by testing for multiple STIs using a single sample.

It should be noted that the methods described herein may be used to diagnose a variety of contagious diseases, including microbial, viral, and cancers. However, for the sake of simplicity and ease of description and example, the following describes methods for diagnosing STIs via extraction-free direct PCR approaches. It should be noted, that STIs may be diagnosed using methods of the invention that target nucleic acids from oncogenic viruses some of which are sexually transmitted (such as IPV). By extension, the methods of the invention may detect one or more cancers (such as lung, head and neck, cervical).

The methods of the present invention provide rapid detection of an STI (i.e., presence of a sexually-transmitted pathogen in a patient) by reducing the number of steps during sample preparation that are typically required with conventional STI detection methods relying on PCR assays. Moreover, methods of the invention that use combined samples, allow testing to be performed concurrently on samples obtained from locations that harbor high concentrations of pathogen, including at different points in time during the course of an infection.

Preferably, samples used in the methods of the invention include one or more non-invasive mucosal membrane swabs and/or fluid sample (e.g., urine and/or saliva). Non-invasive sampling allows for patients to collect samples in their own homes or remote clinics without on-site access to sophisticated laboratory equipment and staff. Advantageously, the extraction-free methods of the invention use proprietary buffer compositions, which allow target nucleic acids from the samples to be preserved and secured for shipping to a laboratory for analysis. Fortuitously, the extraction-free methods of the invention and use of the proprietary buffer compositions also allows for target nucleic acids from the sample to be analyzed at home on an appropriate point-of-care testing device.

As methods of the invention enable sample collection for STI testing at home, the methods provide several advantages over traditional in-clinic testing, including privacy. Thus, methods of the invention may help those, who due to a perceived stigma, are reluctant to seek in-person testing. Moreover, even when not provided in-home, methods of the invention may be used in fairly austere locations, and the samples collected by minimally-trained staff. This finds distinct utility in expanding STI testing beyond centralized locations, e.g., hospitals with specialized staff, to underserved communities.

In general, the workflow for an exemplary method of the invention comprises obtaining a biological sample from an individual. The method of sample collection, as well as the type of samples collected, may depend on the specific sexually transmitted disease to be tested. For example, the samples used in the invention may include one or more mucosal membrane swabs or body fluid samples collected in any clinically accepted manner.

A mucosal membrane sample may include biological material from one or more of a vaginal swab, a cervical swab, a urethral swab, a genital swab, a buccal swab, a throat swab, a nasal swab, ocular swab, and a combination of any thereof. A body fluid sample may be a liquid material derived from, for example, a human or other mammal. Such body fluids include, but are not limited to, mucous, blood, plasma, serum, serum derivatives, bile, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. Samples may also include media containing cells or biological material. Samples may also include a blood clot, for example, a blood clot that has been obtained from whole blood after the serum has been removed.

A swab used in the methods herein may be squeezed or agitated to extract the sample, and in certain aspects, mix it with another portion of a combined sample (e.g., saliva). In certain aspects, a body fluid sample is collected, and a swab placed in the sample for sample preparation.

As previously noted, many current STI testing approaches rely on an initial step of isolating and purifying nucleic acids from a clinical sample. For example, in many prior methods, the application of qPCR for the relative quantification of a nucleic acid of interest is preceded by steps, which may include: (1) the isolation and purification of total nucleic acid from the sample; (2) elution and possible concentration of the material; and/or (3) the use of purified RNA in a reverse-transcription (RT) reaction resulting in complementary DNA (cDNA), which is then utilized for the qPCR reaction. The initial nucleic acid isolation and purification step (i.e., extraction step) required in these methods, prior to undergoing PCR, constitutes a major bottleneck in the diagnostic process, as it remains both manually laborious and expensive, and further increases the chances of accidental contamination and human error.

The present invention provides compositions for processing samples and providing usable DNA for subsequent PCR assays, while eliminating the need for an initial nucleic acid extraction step. For example, a unique buffer composition is used for sample preparation such that, when mixed with the biological sample, it is capable of preparing nucleic acid from the sample which is able to be being directly used for nucleic acid amplification and analysis without the need for initial nucleic acid extraction (i.e., isolation and purification of the nucleic acid).

When there is an insufficient amount of nucleic acid for analysis, a common technique used to increase the amount includes amplifying the nucleic acid. Amplification refers to production of additional copies of a nucleic acid sequence and is generally carried out using polymerase chain reaction or other technologies well known in the art (e.g., Dieffenbach, PCR Primer, a Laboratory Manual, 1995, Cold Spring Harbor Press, Plainview, N.Y.). Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. Primers can be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers can also be obtained from commercial sources such as Operon Technologies, Amersham Pharmacia Biotech, Sigma, and Life Technologies. Amplification or sequencing adapters or barcodes, or a combination thereof, may be attached to the fragmented nucleic acid. Such molecules may be commercially obtained, such as from Integrated DNA Technologies (Coralville, Iowa). In certain embodiments, such sequences are attached to the template nucleic acid molecule with an enzyme such as a ligase. Suitable ligases include T4 DNA ligase and T4 RNA ligase, available commercially from New England Biolabs (Ipswich, Mass.). The ligation may be blunt ended or via use of complementary overhanging ends.

For example, DNA may be synthesized from viral RNA associated with the virus of interest (if present) within the biological sample, via reverse transcription, to thereby produce complementary DNA (cDNA). As generally understood, reverse transcriptases (RTs) use an RNA template and a short primer complementary to the 3′ end of the RNA to direct the synthesis of the first strand cDNA, which can be used directly as a template for amplification (via PCR). This combination of reverse transcription and PCR (RT-PCR) allows the detection of low abundance RNAs in a sample, and production of the corresponding cDNA, thereby facilitating the cloning of low copy genes. Alternatively, the first-strand cDNA can be made double-stranded using DNA Polymerase I and DNA Ligase. Many RTs are available from commercial suppliers. The use of engineered RTs improves the efficiency of full-length product formation, ensuring the copying of the 5′ end of the mRNA transcript is complete, and enabling the propagation and characterization of a faithful DNA copy of an RNA sequence. The use of the more thermostable RTs, where reactions are performed at higher temperatures, can be very helpful when dealing with RNA that contains high amounts of secondary structure.

Digital polymerase chain reaction (dPCR) is a refinement of conventional polymerase chain reaction methods that can be used to directly quantify and clonally amplify nucleic acids strands including DNA, cDNA, or RNA. In dPCR a sample is separated into a large number of partitions and the reaction is carried out in each partition individually, thereby permitting sensitive quantification of target DNA through fluorescence analysis in each partition as opposed to a single value for the entire sample as found in standard PCR techniques.

Droplet Digital PCR (ddPCR) is a method of dPCR wherein the aforementioned partitions consist of nanoliter-sized water-oil emulsion droplets in which PCR reactions and fluorescence detection can be performed using, for example, droplet flow cytometry. The methods for creating and reading droplets for ddPCR have been described in detail elsewhere (see Zhong et al., ‘Multiplex digital PCR: breaking the one target per color barrier of quantitative PCR’, Lab Chip, 11:2167-2174, 2011), but in essence each droplet is like a separate reaction well and, after thermal cycling, the fluorescence intensities of each individual droplet were read out in a flow-through instrument like a flow cytometer that recorded the peak fluorescence intensities.

While compositions and methods of the invention may be used to detect nucleic acid specific to any pathogen, in preferred embodiments, one or more sexually transmitted pathogen is the detection target. Methods of the invention may be used to detect any sexually transmitted infection. Exemplary STIs detected by methods of the invention include bacterial vaginosis, CT, cystitis, NG, hepatitis A, hepatitis B, hepatitis C, herpes (herpes simplex type 1 and 2), HIV, HPV, MPV, lymphogranuloma venereum, molluscum contagiosum, non-gonococcal urethritis, pelvic inflammatory disease, phthirus pubis, syphilis, trichomoniasis, and vaginitis. In certain aspects, methods of the invention detect a plurality of sexually transmitted infections from a single sample. In preferred aspects, methods of the invention detect CT and/or NG infection in a sample.

Methods of the invention may be used to detect oncogenic viruses. Exemplary oncogenic viruses detected by methods of the invention include HPV, Epstein-Barr virus (EBV), hepatitis C and virus (HCV). Methods of the invention are amenable to detecting oncogenic viruses, some of which are sexually transmitted (such as HPV). By extension, the methods of the invention may detect one or more cancers (such as lung, head and neck, cervical).

Accordingly, in certain aspects, methods of the invention may include detecting one or more genetic markers correlated with an elevated risk of cancer. Such genetic markers may be those correlated with a particular pathogen or pathogen variant, for example, those used to discriminate high-risk variants of HPV, including HPV-6, HPV-11, HPV-16, HPV-18, HPV-31, HPV-33, HPV-35, HPV-39, HPV-45, HPV-51, HPV-52, or HPV-68 (See, American Cancer Society, Human Papilloma Virus (HPV), Cancer, HPV Testing, and HPV Vaccines: Frequently Asked Questions (Oct. 22, 2013). Similarly, genetic markers correlated with an elevated risk of cancer may include oncogene sequences and/or a gene mutation sequence (such as KRAS G12C-mutated NSCLC). Exemplary genetic markers include, for example, those associated cervical cancer, such as SC6; SIX1; human cervical cancer 2 protooncogene (HCCR-2); p27; virus oncogene E6; virus oncogene E7; p16INK4A; Mcm proteins (such as Mcm5); Cdc proteins; topoisomerase 2 alpha; PCNA; Ki-67; Cyclin E; p-53; PAIl; DAP-kinase; ESR1; APC; TIMP-3; RAR-β; CALCA; TSLC1; TIP-2; DcR1; CUDR; DcR2; BRCA1; p15; MSH2; RassflA; MLH1; MGMT; SOX1; PAX1; LMX1A; NKX6-1; WT1; ONECUTI; SPAG9; and Rb (retinoblastoma) proteins.

In certain aspects, methods of the invention include targeting one or more endogenous nucleic acids (e.g., genomic DNA/RNA or mRNA transcripts) or gene target(s) of a subject using one or more samples with the stabilizing buffer compositions described herein. Thus, for example, methods of the invention may include as targets one or more human genomic sequence, human transcript sequence, oncogene sequence, and/or gene mutation sequence (such as KRAS G12C-mutated NSCLC). Methods of the invention may include assessing one or more endogenous nucleic acids for a mutation indicative of a disease (e.g., cancer) or other condition (e.g., a predisposition for developing a cancer or cancer progression). Mutations detected using methods of the invention may include, for example somatic mutations, which may be indicative of a cancer/tumor or minimal residual diseases. In certain aspects, methods of the invention include assessing the methylation status of one or more target nucleic acid. DNA methylation plays a role in regulating gene expression, and aberrant DNA methylation is associated in many diseases, including cancer. DNA methylation profiling, including longitudinal profiling, is a valuable diagnostic tool for detection, diagnosis, and/or monitoring of cancer. For example, specific patterns of differentially methylated regions and/or allele specific methylation patterns may be useful as molecular markers for non-invasive diagnostics using target nucleic acids obtained using the nucleic acid extraction-free methods of the invention.

In certain aspects, target nucleic acids are used to monitor or assess the progression of a disease or condition in a subject, e.g., a cancer or infection. Assessing a disease in accordance with methods of the invention can include one or more of predicting disease severity, determining a diagnosis or stage of disease progression, classifying cancer type, and predicting a drug response. Certain methods of the invention can include obtaining target nucleic acids from a sample that are used to diagnose a tumor before the tumor is visible. This allows earlier treatment than is provided by existing modalities of diagnosis.

Methods of the invention may be used to provide a longitudinal assessment of a subject's disease or condition. For example, a longitudinal assessment may include obtaining samples from a subject at multiple points in time and amplifying target nucleic acids using the nucleic acid extraction-free methods of the invention. The amplicons assessed from multiple time points may be used to assess, for example, progression of a cancer, development of a particular subtype of cancer, minimum residual disease, likely risk of metastasis, any benefit in further monitoring, changes in methylation status or pattern, gene expression patterns, and the like.

Compositions and methods of the invention for the detection of sexually transmitted infection include the use of one or more PCR assays, such as ddPCR, of target nucleic acids obtained from a mucosal membrane swab(s) sample and/or a bodily fluid sample. Furthermore, in some embodiments, the step of performing the one or more PCR assays includes using a primer-probe set specific to ribonuclease P (RNP).

In addition to diagnosing an individual as having been infected with an STI, inventive methods may further include the step of determining the severity of the infection based on the target nucleic acid quantity in the sample. For example, methods of the invention are useful to assess viral or bacterial load, which can be directly correlated with disease severity and/or progression. In some embodiments, methods may further include the step of comparing target nucleic acid quantities in a plurality of combined biological samples obtained from the patient at successive time points and determining disease progression based on increases or decreases in the target nucleic acid quantities over time. Methods of the invention can also be used to predict disease outcomes and/or severity based on the target nucleic acid quantity.

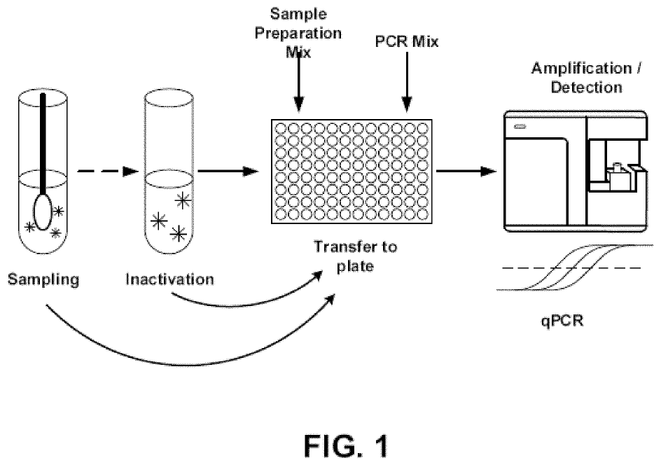

FIG. 1 shows a schematic overview of an extraction-free, real-time qPCR test intended for the qualitative detection of nucleic acid from one or more sexually transmitted pathogens in biological specimens (e.g., swab and/or bodily fluid samples) collected and processed via unique buffer compositions of the present invention. In certain aspects, a body fluid sample is collected in an acceptable vessel. A swab, spatula, brush, or similar device is used for the collection of mucosal membrane sample and then placed within the vessel containing the body fluid sample. The swab can be squeezed or agitated to extract the mucosal membrane sample and mix it with the body fluid sample. The vessel may include a unique buffer composition of the invention, or it may be added after the combined sample. In certain aspects, the buffer composition can be used for sample preparation and/or a transport medium.

After collecting the samples and providing them with the unique buffer composition, viral particles and/or bacteria may be inactivated either through heating or by direct lysis in the buffer. The inactivated samples can then be used for downstream qPCR diagnostic testing without the need for the additional nucleic acid extraction step (isolation and purification) that conventional approaches rely on.

In certain aspects, the prepared sample may be transferred to a PCR-plate (96/384-well) format in which cDNA synthesis by RT (if needed) and/or detection by qPCR may take place.

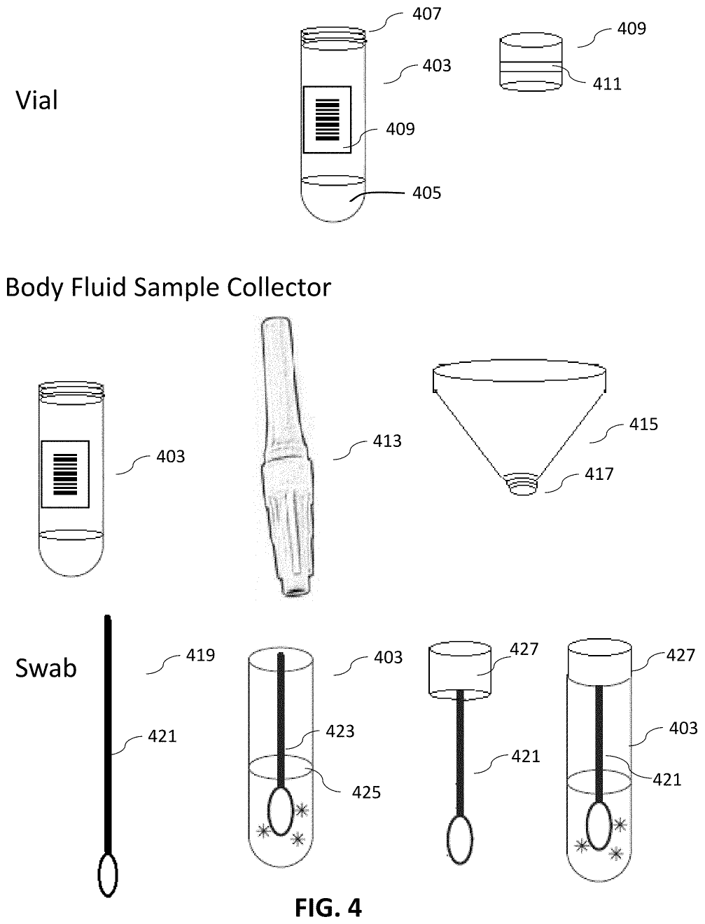

FIG. 4 shows certain components used in the methods of the invention. In certain aspects, one or more of the components can be provided as part of a diagnostic kit, along with instructions for use. As shown, the methods and kits of the invention may include a vial 403. In certain aspects, the vial is provided with a buffer composition 405. The buffer composition is for example, a pathogenic nucleic acid transport buffer as disclosed herein. In certain kits and methods of the invention, the vial 403 is provided pre-filled with the buffer composition 405. Alternatively, the buffer composition is added to the vial before or after sample collection.

Preferably, the vial is at least 1.5 mL such that it can accommodate least one swab sample and/or a body fluid sample, and a buffer composition of the invention. For example, samples may be collected in a centrifuge tube, such as the screw cap cryovial. An exemplary vial includes a barcode 407, which can be used to track individual vials and/or collected samples. Vials useful in connection with the presently disclosed invention include, polypropylene cryovials, such as the NEST Scientific USA (NJ, USA) 1.9 mL 2D Barcoded cryovials.

In certain aspects, the vial includes a thread 407 or other means for affixing a cap, lid, funnel, and/or a body fluid sample collection aid (shown as a saliva collection aid in FIG. 4). In certain aspects, the thread 407 or other affixing means is used to affix a cap 409 to the vial to seal the sample for transport and/or storage. In certain aspects, the cap 409 includes a compartment or pouch 411. Affixing the cap 409 on the vial causes the compartment to perforate or otherwise release a buffer composition from inside the compartment or pouch 411 into the vial 403.

In certain aspects, methods and kits of the invention include a means for collecting a body fluid sample from a subject. In some methods and kits, the subject merely provides a body fluid sample in a sterile vial 403. Alternatively, a sample collection aid 413, e.g., a saliva collection aid, or funnel 415 is provided to facilitate collection. The sample collection aid 413 or funnel 415 may include a means, such as screw threads 417, for coupling the collection aid/funnel to the vial during fluid collection. Alternatively, the funnel or collection aid is integrated into the vial to form a single unit.

Preferably, when provided as a diagnostic kit, the collection aid/funnel is pre-attached to the vial. The collection aid/funnel may include a means for sealing the sample, such as a lid or cap. Alternatively, the collection aid/funnel can be removed, e.g., through a thread and screw attachment means. Once removed, the collection aid/funnel can be replaced by a cap or lid for sealing the sample in the vial.

A collection aid 413 or funnel 415 may include a pouch or compartment that includes a buffer composition, such as a transport buffer as disclosed herein. The pouch or compartment may release the buffer during sample collection. For example, the pouch or compartment may be integrated within a lid or cap for the funnel/collection aid, such as that used with in the OME-505 collection kit, DNA Genetek, Inc., Ottawa, Canada. Closing the lid or cap causes a compartment to perforate, thereby releasing the buffer into the vial with the sample.

Methods and kits of the invention may include or use a swab for collecting one or more mucosal membrane samples. In certain aspects, the swab 419 includes a handle 421, which is held while a sample is being obtained from a subject. The handle 421 may include a break point. After the sample is obtained, the handle is snapped at the break point, which shortens the length of the handle. The swab with shortened handle 423 is thus short enough to fit within the vial 403. As shown, the level 425 of the buffer (and any fluid sample) in the vial is sufficient to cover the swab. However, the level 425 of the fluid sample/buffer need not cover the swab. Rather, it is only necessary that the fluid sample/buffer are in an adequate quantity such that and swab and can be mixed in the vial.

Alternatively or additionally, the swab 421 is coupled to a cap 427. The cap 427 can be coupled to the vial 403 after sample collection to seal the sample for transport, storage, and/or processing. As shown, when the cap 427 is affixed to the vial 403, the swab is positioned within the buffer in the vial.

In preferred aspects, the buffer composition is provided in a pre-filled vial or as part of another component of the kit, e.g., a cap as described herein. By providing the buffer in a pre-measured volume in a manner than can be easily added to the sample by a subject, exemplary kits of the invention allow a subject to provide a sample at home. By adding the pre-measured, novel transport buffer compositions of the invention to the sample, the subject can provide the sample at home or any other convenient location and send it via post to a laboratory for analysis.

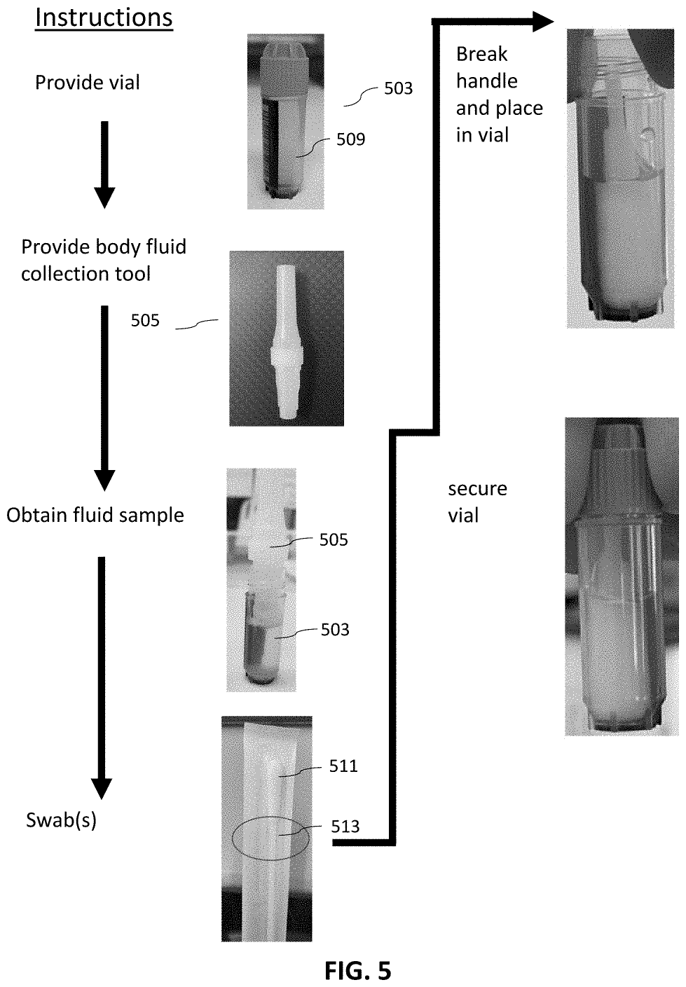

FIG. 5 details select components of a kit of the invention used to detect a target nucleic acid (e.g., one indicative of a sexually transmitted pathogen) in a sample. The kit includes instructions, which include the steps necessary to obtain a sample, e.g., swab(s) and/or body fluid sample(s). The instructions outline that a vial 503 is provided to a subject along with a sample collection tool, such as a swab, brush, spatula, paddle, or similar to obtain a mucosal membrane swab and/or tools such as a fluid sample collection aid 505. As shown, in certain kits of the invention, the vial 503 comes pre-filled with a transport buffer 509, as described herein.

In the kit shown, the subject uses the provided body fluid sample collection aid 505 to provide a sample (e.g., urine or saliva) to the vial. As shown, the fluid sample collection aid 505 is shaped to fit securely in the opening of the vial 503 to facilitate sample collection.

The kit also includes a swab 511, which is used to obtain a mucosal membrane swab (e.g., a vaginal swab). The handle of the swab includes a break point 513. After the swab is used to obtain a sample, the handle is snapped at the break point. The shortened swab is placed into the vial with the body fluid sample and buffer. The vial is then sealed with a cap for storage or transport. In certain aspects, the kit includes materials for a subject to mail the combined sample to a lab for analysis.

Although the kit shown in FIG. 5 includes provisions to obtain a body fluid sample, as described herein, the present invention contemplates kits that do not require body fluid samples, kits that require multiple body fluid samples, and kits that require multiple mucosal membrane swabs that may be combined in certain methods of the invention.

In certain aspects, the kit includes one or more primers, at least one of which is used for amplification and/or detection of a target nucleic acid in the sample.

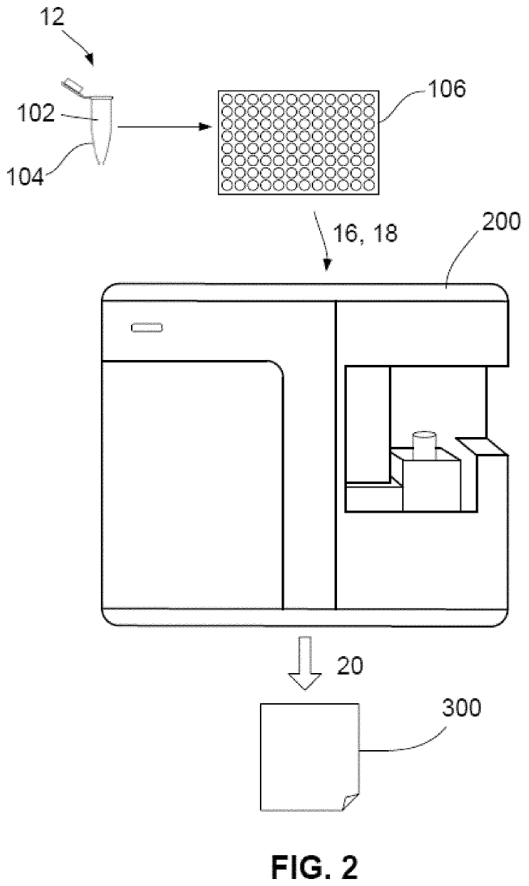

FIG. 2 shows a mucosal membrane sample 102 for STI testing that has been collected from a patient and loading of the sample into an instrument 200 capable of performing one or more assays on the sample to determine whether one or more target nucleic acids associated with at least one sexually transmitted pathogen is present in the sample. As will be described in greater detail herein, the sample 102 may be contained within a suitable container 104 that is obtained 12 from a patient. In certain aspects, the patient is suspected of having one or more STIs, e.g., by displaying symptoms or due to reported sexual contact with a person suspected of having an STI. Alternatively, methods of the invention may be used for ongoing monitoring of patients and/or for routine STI checkups.

Samples may be collected and stored in their own container, such as a centrifuge tube such as the screw cap cryovial. Preferably a 1.9 ml cryovial with screw cap is used. A swab or similar tool with a proximal breakpoint is used, which allows the swab to be inserted into the tube after for sample collection. The screw cap is important to prevent contamination. The standard size of cryovial allows direct sample storage without additional sample transfer. A funnel or sample collection aid may be used to facilitate collection of body fluid samples, if desired.

FIG. 2 further illustrates loading of the sample 102 into a PCR-plate 106, in which sample preparation may take place (introduction of the sample to the unique buffer and/or PCR mix), at which point the plate 106 may then be introduced into an instrument 200 capable of performing one or more PCR assays on the sample 102 to determine whether one or more target nucleic acids associated with at least one sexually transmitted pathogen is present in the sample. In particular, the instrument 200 may be configured to provide any one of the prior steps of method, including, but not limited to, detection of target DNA and/or RNA, reverse transcribing any target RNA to produce cDNA, amplification of target DNA/cDNA (operation 16), analysis of data from the amplification step (operation 18), and generation of a report 300 providing information related to the STI evaluation (operation 20).

Accordingly, the instrument 200 is generally configured to detect, sequence, and/or count the target nucleic acid(s) or resulting fragments. In this instance, where a plurality of fragments is present or expected, the fragment may be quantified, e.g., by qPCR. The resulting report 300 may include the specific data associated with the assay, including, for example, patient data (i.e., background information, attributes and characteristics, medical history, tracing information, etc.), test data, including whether the sample tested positive or negative for one or more target pathogens, and, if positive, further metrics, including disease progression and predicted disease outcome.

### EXAMPLES

The following examples provide exemplary protocols for detection of target, pathogenic nucleic acids in accordance with methods of the present invention. The following examples show, among other facets of the invention, that the methods are successfully able to provide usable DNA for pathogen testing without a nucleic acid extraction step. Further, as shown, the methods of the invention are applicable to samples obtained via a mucosal membrane swab, body fluid samples, and a combined swab and body fluid sample. Moreover, the methods of the invention are able to detect both viruses and bacteria from a sample, and also able to detect and discriminate a plurality of sexually transmitted pathogens using a single test.

### Example 1—Extraction-Free Methods in Swab, Body Fluid, and Combined Samples

Extraction-free PCR relies, in part, on the efficacy of proteinase K (PK) digestion, which would otherwise degrade a desired sample of DNA or RNA. To optimize for PK activity in either a swab or saliva matrix, a variety of buffer components were tested. This is particularly important for swab samples. Unlike body fluid samples (e.g., urine and saliva), which one is able to collect and transport as a raw sample, swab samples should be stored in a transport medium, e.g., a viral and/or bacterial transport medium. However, for many sexually transmitted pathogens Conventional swab samples in transport usually require a nucleic acid (e.g., DNA or RNA) extraction step for testing.

The present inventors tested a variety of buffer components, a viral transport medium, and a commercial swab collection device-OR100 (DNA Genotek) for extraction-free PCR. Negative swab samples were collected from healthy volunteers and put into each solution. Samples then were spiked into heat-inactivated SARS-CoV-2 virus, mixed with PK by aliquoting sample into a 96-well plate pre-filled with either a mix of saliva preparation buffer (see below) and PK (Promega) for saliva samples or PK alone for swab samples. For saliva samples (SalivaFAST), 30 μL from a single saliva sample was mixed with 5 μL saliva preparation buffer and 5 μL PK in each well of the plate. For swab samples (SwabFAST), 35 μL from a single swab sample was mixed with 5 μL PK per well. The prepared sample plate was then placed on a digital microplate shaker at 500 RPM for one minute, then on a thermal cycler at 95° C. for five minutes for heat-inactivation.

Swab samples in PBS, viral transport medium, and OR100 did not generate positive signals at N1 region. Among the positive signals, the contrived swab sample in Tris-Borate-EDTA (TBE) buffer produced the strongest quantification cycle (Cq) value, which comprise the buffer component for the viral transport buffer of the invention. Similarly, a variety of buffer components, raw saliva, and a commercial saliva collection device-OM505 (DNA Genotek) were tested for extraction-free PCR. Contrived saliva samples in OM505 did not generate positive signals at N1 region. Among the positive signals, the contrived saliva sample in Tris (2-carboxyethyl) phosphine (TCEP) buffer condition produced the strongest Cq value, which is used to improve PK efficacy in the SalivaFAST protocol.

Accordingly, the nucleic-acid extraction free methods of the invention using the transport buffers described herein, permit stable storage and detection of target nucleic acids from both swab and fluid samples.

The present inventors then tested the relative efficacies of mucosal membrane swab samples and body fluid samples. Anterior nasal swab (ANS) samples and saliva samples were compared for the detection of the SARS-CoV-2 virus. Briefly, an ANS sample was collected with DNA Genotek's OR-100 device (SwabClear™), and a saliva sample was collected using DNA Genotek's OM-505 device (SalivaClear™) from the same patients. Samples were run to detect SARS-CoV-2 virus in accordance with the manufacturer's instructions.

Although most paired samples showed consistent results (detection in both or non-detection in both) between ANS and saliva samples, discordant results between the two types of specimens were observed in some paired samples (i.e., detection of SARS-CoV2 in one specimen but non-detection in the other). Based these clinical observations, it was hypothesized that the abundance or clearance of SARS-CoV-2 or other respiratory viruses can vary in nasal cavity vs. in saliva across individuals or at different points of time during the course of the infection or disease. Thus, tests relying on only one specimen site can mean missing some positive cases. This has application to the STI detection methods of the invention, as many sexually transmitted pathogens are differently detectable across various sample types, including over the course of an infection.

The present inventors further produced experimental results showing that methods of the invention are able to use combined swab and body fluid samples. Sixteen human participants spit saliva samples into 50 ml falcon tubes. A flocked nasopharyngeal swab was used to collect anterior nares swab (ANS) samples from the same participants. One saliva sample from each patient was used in the RNA-extraction free qPCR protocol to detect a SARS-CoV-2 infection. The nasal swabs were placed swab down in falcon tube holding a second saliva sample from each participant. The swabs were squeezed to extract the ANS sample and mix it with the saliva. The combined saliva and ANS samples underwent the same RNA-extraction free qPCR protocol as the saliva samples.

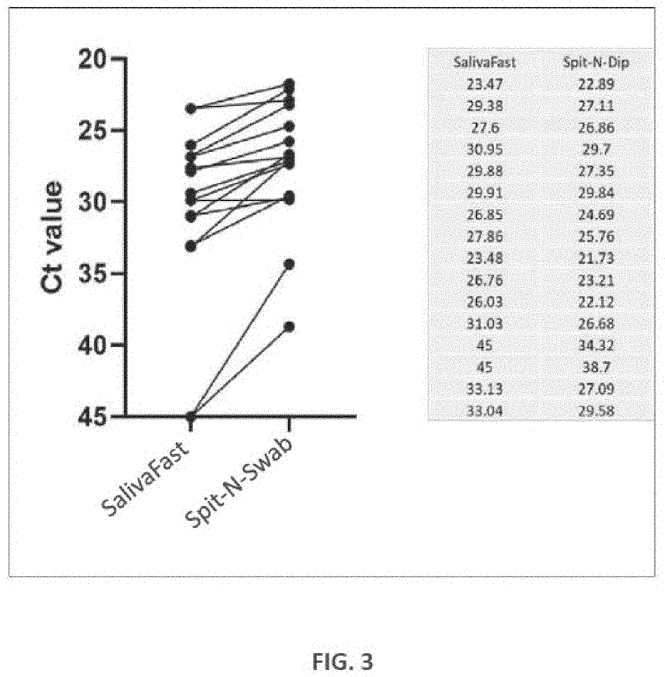

FIG. 3 provides the qPCR results as cycle threshold (Ct) values, which indicate how much SARS-CoV-2 virus was detected in the sample. The paired results for the saliva-only samples are provided as “SalivaFast” and the combined samples as “Spit-N-Dip”. This data shows a significant improvement of enriched viral abundance (shown as lower Ct value) in the mixed ANS-Saliva specimens when compared to testing using only saliva using the same testing protocol.

Thus, for certain pathogens and/or sample types, a combined mucosal membrane swab and body fluid sample provides more sensitive results when compared to samples obtained from a single source. Consequently, the presently disclosed methods can be used for STI detection tests that combine one or more mucosal membrane swabs and one or more body fluid sample to maximize the chance of detection of sexually transmitted pathogens of interest among diverse populations and at different points of time during the infection or disease course.

### Example 2—Exemplary Protocols for STI Detection Using Methods without a Step of Nucleic Acid Extraction

The present disclosure provides this exemplary protocol for a nucleic acid extraction-free method of performing STI detection for a CT and/or NG infection. However, the methods of the invention may be used to detect nucleic acids from any other sexually transmitted pathogen.

### Swab Sample Collection:

Swab collection devices include: a 1.9 ml Nest tube filled with 1 ml a unique buffer composition specific to swab samples (hereinafter referred to as Swab Transport Buffer), which will be used as the container of the swab sample; and at least one swab will be used to swab a patient's mucosal membrane(s) and later be placed inside the tube filled with the Swab Transport Buffer.

The swab(s) may be collected under the supervision of a trained healthcare worker designated by the organization overseeing the collection site. Alternatively, a kit may be sent to a patient at their home or other remote facility. A healthcare worker supervising the collection or the patient obtaining the sample should clean hands with alcohol-based sanitizer or fragrance-free soap and water. Before collection, patients are provided instructional materials. A patient may provide patient information, including name, date of birth, and additional information required. A healthcare worker may ask the patient to review a study consent form to opt in or out of the study (provided by Ovation). Lastly, a healthcare worker will scan a pre-printed barcode label to tie it to the patient information that is already collected, then place the label on the tube that will be used by the patient for sample collection.

For collection, the cap of the Nest tube is removed, and a mucosal membrane of interest for the particular test is swabbed ten times. The handle of the swab is broken inside the tube at a proximal breakpoint. The cap of the Nest tube is replaced with the swab(s) inside and securely tightened. If there is any sample spill during the collection process, an alcohol wipe or equivalent to wipe the outside of the tube is used to prevent contamination. The sample will then be placed in an individual bag under room temperature before being transported to the lab.

### Sample Receiving and Accessioning in Lab:

Swab samples are transported to the lab. Samples will be removed from bags and visually examined by the accessioning supervisor at the receipt desk for any leakage or damage. Samples passed the pre-screening step by the supervisor are moved to the desktop used by the accessioning team. Samples failed the pre-screening step are set aside for further investigation. Accessioners will scan the barcodes on the Nest tubes and examine patient information and consent status shown on a computer screen via a laboratory information management system (LIMS). Tubes with complete patient information in the LIMS and have no leakage (i.e., qualified samples), are placed in a rack. The positions of the samples in the rack should match assigned positions in the LIMS. Disqualified samples are placed in another rack and set aside for further investigation by the accessioning supervisor. The rack of samples may then be placed on a platform rocker in hold position @ 600 rpm until a medical lab scientist (MLS) from the sample preparation team fetches the samples.

### Swab Preparation Buffer:

As part of sample preparation, the swab sample will be mixed with a unique buffer composition prepared specifically for swab samples (referred to herein as Swab Preparation Buffer). Preparation of the Swab Preparation Buffer includes use of at least the following equipment: Biosafety cabinet or laminar flow hood (workspace capable of maintaining an aseptic environment); individual, sterile wrapped pipettes, pipette tips, such as 10 and 25 mL; pipette aid; pipettor, 1 mL or 200 μL and corresponding tips; 50 ml sterile, nuclease-free Falcon tubes; Eppendorf repeater (50 mL capacity); 1.9 ml Cryovial tubes, Nest; Nest tube racks; and screw cap tube decapper equipment, Brooks Life Sciences.

The preparation of the Swab Transport Buffer further includes use of at least the following reagents/components:
10×TBE Buffer (Tris-Borate-EDTA, pH 8.2-8.4), Sterile, DNase-, RNase- and Protease-Free grade, Fisher BioReagents, catalog number BP133320, 20L;
RNase inhibitor, human placenta, 40,000 units/ml, Sterile, DNase-, RNase-Free grade, New England Biolabs, catalog number M0307L, 10,000 units, 250 ul/tube;
Amphotericin B solution, 250 μg/ml in deionized water, sterile, Sigma-Aldrich, catalog number A2942, 100 ml (or similar antifungal at an appropriate concentration to prevent fungal contamination and growth);
Penicillin-Streptomycin Solution, 100×, a mix of Penicillin (10,000 IU) and Streptomycin (10,000 μg/ml) in a 100-fold working concentration, Sterile, Corning, catalog number 30-002-CI (or similar antibiotics at an appropriate concentration to prevent bacteria contamination and growth;
Nuclease-free water, Sterile, Millipore/Sigma, W4502, DNase-, RNase- and Protease-Free grade; and
Disinfectant, such as 70% ethanol.

Preparation of the ingredients includes at least the following steps: clean work surface with appropriate disinfectant; disinfect reagent bottles prior to placing on work surface; aliquot 10×TBE Buffer, 500 ml/bottle in Corning 500 ml sterile bottle, store at RT; aliquot nuclease-free water, 894.95 ml/bottle in Corning 1L sterile bottle, store at RT; aliquot Amphotericin B solution 4 ml/tube (in 5 ml sterile Corning tube), store at −20C; aliquot Penicillin/Streptomycin, 1 ml/tube (in sterile Eppendorf tubes), store at −20C; and Record lot information and preparation in a laboratory-controlled notebook.

Preparation of the Swab Preparation Buffer includes at least the following steps:

1. Clean work surface with appropriate disinfectant;

2. Disinfect reagent bottles (aliquot, except RNase inhibitor) prior to placing on work surface;

3. For example, to prepare 1L transport buffer:
3.1. bring 1 bottle of nuclease-free water (894.95 ml/bottle);
3.2. using a sterile 50 ml falcon tube, add 100 ml of 10×TBE Buffer;
3.3. using a sterile pipette, add 50 μl of RNase inhibitor; and
3.4 thaw a tube of Amphotericin B solution and a tube of Penicillin/Streptomycin, using a sterile pipette, aseptically add 4 ml of Amphotericin and 1 ml of Penicillin/Streptomycin to the bottle.

4. Record lot information and preparation in a laboratory-controlled notebook;

5. Assign laboratory appropriate identification (e.g. lot number);

6. Cap the tube securely and mix thoroughly by inverting the tube;

7. Withdraw 100 ul of medium for QC sample;

8. Label the bottle as:
SWAB TRANSPORT BUFFER
Lab ID: (Insert laboratory appropriate identification, such as STB2 as Summit Buffer 2)
DOM: (Insert current date of manufacture)
Expires: (Insert date 1 month after manufacture date)
Store at 2-8C.

9. Store at 2-8C., until dispensed into aliquots;

10. Aliquot 1 mL of prepared Swab Preparation Buffer into individual sterile 1.9 ml screw-capped tubes (Nest) using Eppendorf repeater (50 mL capacity) and Brooks decapper;

11. Perform sterility check; and

12. Store tubes and any buffer remaining in the bottle at 2-8C.

### Sample Preparation:

An MLS from the sample preparation team will fetch the racks of accessioned samples on the rocker and bring them into the sample preparation room to prepare them for testing. They will bring prepared 96-well Sample Prep Plate (SPP) containing 10 μL/well of a Sample Prep Mix (SPM). The SPM contains the Sample Preparation Buffer and a protease (Proteinase K). In particular, the 96-well SPP contains 10 μL SPM (5 μL Sample Preparation Buffer and 5 μL Proteinase K (Promega))/well, dispensed into each well using a multichannel equalizer or Viaflow (Integra). The samples are decapped with a semi-automated 6-channel decapper (Brooks) or automated 48-format decapper (Brooks) inside the biosafety cabinets. Caps will be temporarily placed on the cap carrier rack when using the 6-channel decapper. Approximately 30 μL of the sample are transferred from the tubes in the 48-well rack using the E1-ClipTip electronic multichannel (8-channels) equalizer to the 96-well SPP containing the 10 μL SPM and pipetting well. Two 48-well racks of samples will fill one 96-well SPP. Samples are recapped (6 at a time if using the 6-channel decapper or 48 at a time if using the automated 48-format decapper). The samples and SPM are mixed well by placing the plates on the digital microplate shaker@500 RPM for 1 minute. The plate is placed on the miniAmp 96-well PCR instrument at 95° C. for 5 minutes, and 4° C. on hold. The entire racks of samples are then brought to the temporary sample storage area. Any of the samples that require repeat testing will be identified from the temporary sample storage area. Repeat testing is only allowed one time. If failed, request a new sample. Store left-over samples in −80° C. for future use.

### PCR Reagent Preparation and Plate Setup:

A plate containing a PCR master mix (herein referred to as a PCR Master Mix Plate (PMMP), includes 12.5 μL of PCR master mix dispensed into each well of the plate using a multichannel equalizer or Viaflow (Integra) on to a 96- or 384-well plate. The PCR master mix is composed of 10 μL Luna Universal Probe One-Step Reaction Mix, 1 μL Luna Warmstart RT enzyme Mix, and 1.5 μL of pathogen-specific/RNP primer/probe. The 1.5 μL pathogen-specific/RNP primer/probe will be made as: 66.7 μM working stocks of the pathogen-specific and RNP primers and 1.7 μM FAM-labeled pathogen-specific and ATTO-647 labeled RNP probe by adding 50.25 μL of each 100 μM primers and probe stock to 524 μL IDTE buffer (pH7.5). Alternatively, where multiple pathogens are to be detected in a single sample, the 1.5 μL pathogen-specific/RNP primer/probe will be made as: 66.7 μM working stocks of the pathogen-specific and RNP primers and 1.7 μM of differently labeled pathogen-specific probes (e.g., a Rox labeled probe for NG detection and a Fam labeled probe for CT detection) and the ATTO-647 labeled RNP probe by adding 50.25 μL of each 100 μM primers and probe stock to 524 μL IDTE buffer (pH7.5).

The MLS in the molecular team will place a 96- or a 384-well PMMP into their individual PCR workstation and add 7.5 μL of treated sample from the Sample Preparation Step to each designated well of the PMMP. The treated sample is then mixed with the PCR master mix by pipetting, taking care to avoid introducing bubbles. The MLS may add 7.5 μL of positive control (e.g., from inactivated CT/NG swabs like those from Microbiologics and Seracare), and negative control, and no-template control (NTC—water) to designated PCR wells for the controls (1 positive control, 1 negative control, and NTC per plate) and mixes by pipetting, avoiding introducing bubbles. The MLS then places a transparent plastic qPCR film on the PMMP and seals the film with a plate sealer and spin briefly to remove bubbles with a plate spinner.

### PCR Thermal Profile (Amplification Area):

Load the plate into a Bio-Rad CFX or a QuantStudio PCR machine, Open master file run the following thermocycler conditions:
1. Step 1: 55° C. 10 minutes, 1 cycle;
2. Step 2: 95° C. 1 minute, 1 cycle; and
Step 3: 95° C. 10 sec, 60° C. 30 sec (+plate read at both FAM channel for CT target and/or Rox channel for NG target & Cy5 channel for RNP target) for 40 cycles.

### Data Interpretation (BioRad CFX Opus 96-Well Format) (Saliva/Mucosa Testing):

The Bio-Rad CFX reports Cq values, in which the Cq value files (csv file) are exported from the PCR machine to the OvDx LIMS. Interpretation of the Cq values (DETECTED, NOT DETECTED, and INVALID) will be exported to the OvDx LIMS according to the following criteria:

| | Cq: CT | Cq: NG | Cq: RNP |
|---|---|---|---|
| | (FAM channel) | (Rox channel) | (Cy5 channel) |
| (NG positive) | | ≤25 | Any number |
| DETECTED | | | or NaN |
| (NG negative) | | >25 | ≤25 |
| NOT DETECTED | | | |
| (CT positive) | ≤25 | | Any number |
| DETECTED | | | or NaN |
| (CT negative) | >25 | | ≤25 |
| NOT DETECTED | | | |
| INVALID | >25 | >25 | >25 |

If CT/NG is detected, the result is valid ad returns a “DETECTED” regardless of value for RNP. If CT/NG is NOT detected and RNP is ≤25, then return a result of “NOT DETECTED”. If RNP Cq value>25 and if CT/NG>25, then the sample is requeue for retesting. After retesting, if the RNP is still>25, then the provider must be contacted to collect another sample. NaN=not a number.

### Quality Assurance and Batch Release:

The Lab Supervisor will examine the controls, including: positive control, which should be positive for NG/CT, but negative for RNP targets; Negative control, should be negative for NG/CT, but positive for RNP targets; and NTC control should be negative for both NG/CT and RNP targets. The Lab Supervisor will further spot check run and estimate positive-negative results ratio. The Medical Director will release the batch and sign off on the report after further examination.

Samples Placement after PCR Testing):

Samples with INVALID results will be identified in the temporary sample storage area (fume hood 1). Repeated testing will be performed on these samples starting from Step III (Sample Preparation). Samples with verified results will be stored at −80° C. PCR plates will be moved to the disposal area (fume hood 2) as biohazards.

### Example 3—STIFast Spiking Experiments

Using protocols like that provided above, the present inventors developed STIFast, which is a nucleic acid extraction-free method of detecting sexually transmitted pathogens from a sample. In order to test the ability of STIFast to detect sexually transmitted infections, the present inventors undertook a series of spiking experiments showing the ability of the methods to detect the presence of either CT or NG in a sample.

In the following proof of concept experiments, CT/NG samples were provided in an inactivated swab. The CT/NG swabs included the Helix Elite™ CT/NG control swabs produced by Microbiologics, Inc. These swabs include CT and NG bacteria at a concentration of 1×10³-5×10³. In other trials, the CT/NG swabs included ACCURUN molecular controls by Seracare, which provides CT/NG nucleic acids in a proprietary concentration. Samples for testing were produced by spiking the pathogen transport buffer with a desired control material.

Table 1 below details the CT and NG probes and primers used in spiking assays.

TABLE 1

| | | | Final |
|---|---|---|---|
| | | | Concentration |
| Name | Oligonucleotide Sequence (5′-3′) | Fluor | (uM) |
| NG | GGATACGACGTAACCTTGACTATGG | | 66.7 |
| Forward | (SEQ ID NO: 1) | | |
| Primer | | | |
| NG | CCGATGTAGAAGACCCTTTTGC (SEQ ID NO: 2) | | 66.7 |
| Reverse | | | |
| Primer | | | |
| NG | CAACGCCAAAGACTACGGTGTAGCACAG [BHQ2A] | Amino | 16.7 |
| Probe | (SEQ ID NO: 3) | C6 + Rox | |
| CT | GGATTGACTCCGACAACGTATTC (SEQ ID NO: 4) | | 66.7 |
| Forward | | | |
| Primer | | | |
| CT | ATCATTGCCATTAGAAAGCGCATT (SEQ ID NO: 5) | | 66.7 |
| Reverse | | | |
| Primer | | | |
| CT | TTACGTGTAGGCGGTTTAGAAAGCGG [BHQ1A] | 6 FAM | 16.7 |
| Probe | (SEQ ID NO: 6) | | |

Ribonuclease P (RNP) target control primers and probes as reported previously for
COVIDFast ™
Chlamydia trachomatis (CT) and Neisseria gonorrhoeae (NG)

In a first set of singleplex assays, samples were spiked using the combined NG/CT controls (i.e., the Helix Elite™ CT/NG control swabs or the CT/NG control nucleic acid) at varying concentrations in TE buffer. Combined samples using both the Helix Elite™ CT/NG control swabs or the CT/NG control nucleic acid were also prepared. RNP was used as a negative control. “Random patient specimen” samples were also prepared using swabs from a subject without a CT or NG infection.

A first set of singleplex assays was performed using the NG and RNP primers and probes and second set of singleplex assays was performed using the CT and RNP primers and probes.

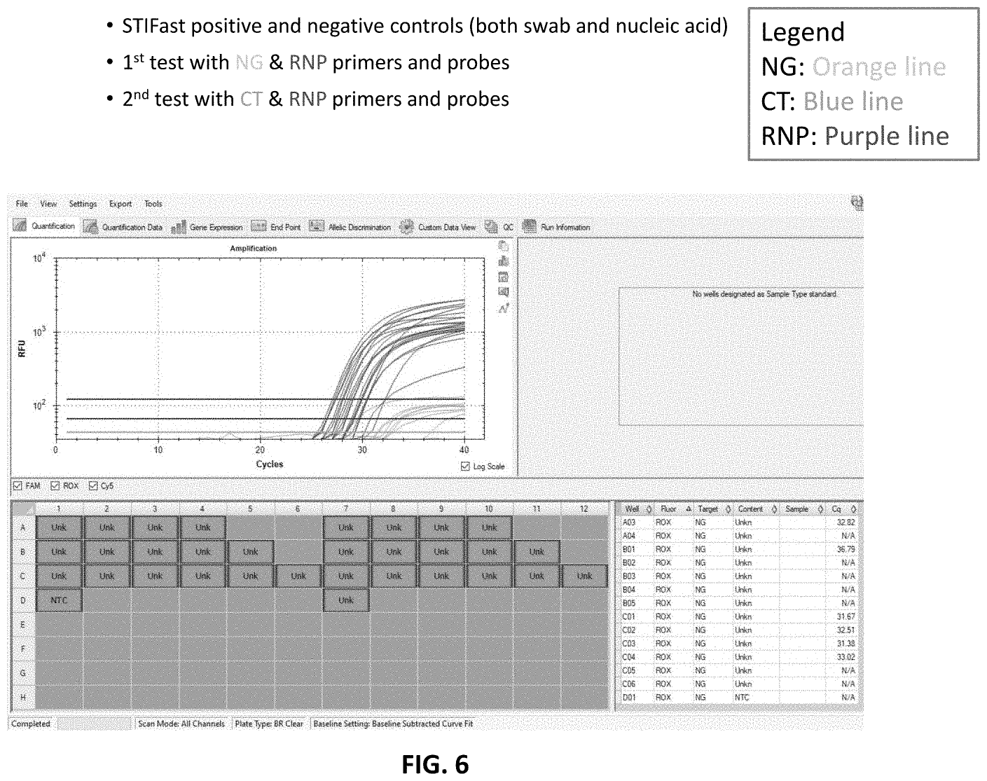

FIG. 6 provides the qPCR readouts from these assays.

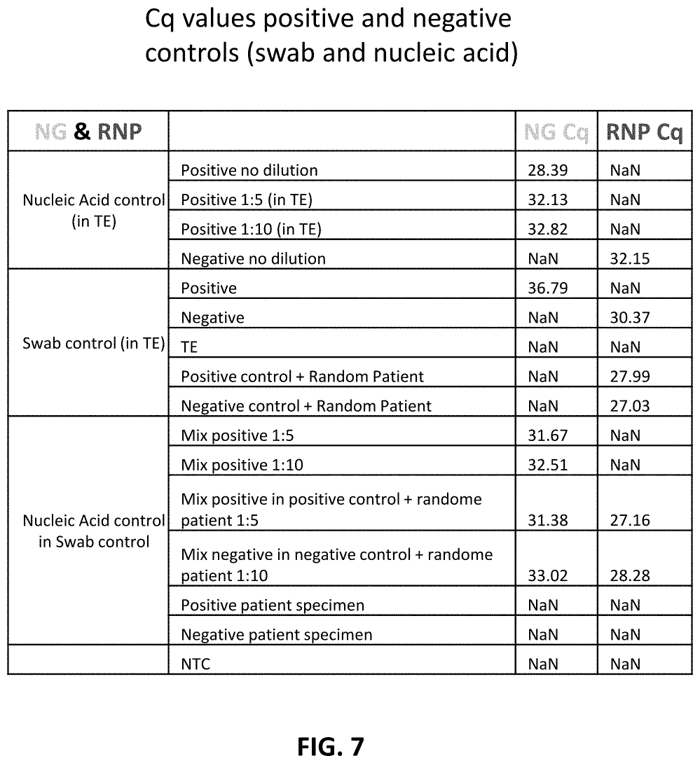

FIG. 7 provides the qPCR Cq data and components used in the NG singleplex assays. As shown by the Cq values, samples made using the CT/NG nucleic acid control provided detectable, target NG DNA. The lack of corresponding RNP negative control signals indicates that only NG target DNA was being detected. Similarly, samples made using Helix Elite™ CT/NG control swabs provided detectable, target NG DNA. Indicating that methods of the invention can be used to detect STIs using a mucosal membrane swab sample. The lack of detectable NG signal upon addition of the non-infected patient samples (and the corresponding RNP signal) indicates that only target NG DNA was detected. Similarly accurate results were obtained when both the Helix Elite™ CT/NG control swabs and the CT/NG control nucleic acid were used to produce a combined sample. Moreover, the Cq values for the combined sample were higher than for either the Helix Elite™ CT/NG control swabs and the CT/NG control nucleic acid samples alone.

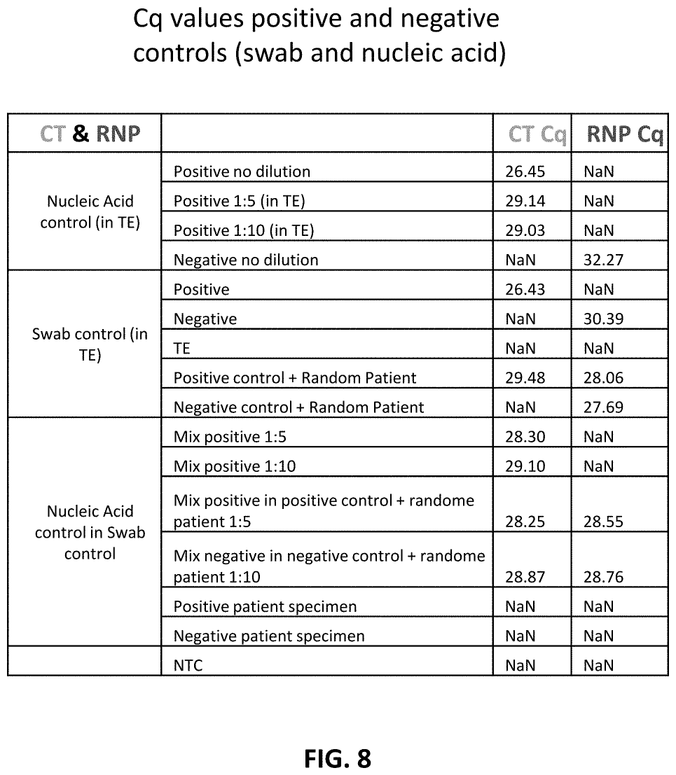

FIG. 8 provides the qPCR Cq data and components used in the CT singleplex assays. As shown by the Cq values, samples made using the CT/NG nucleic acid control provided detectable, target CT DNA. The lack of corresponding RNP negative control signals indicates that only CT target DNA was being detected. Similarly, samples made using Helix Elite™ CT/NG control swabs provided detectable, target CT DNA. Indicating that methods of the invention can be used to detect STIs using a mucosal membrane swab sample. The lack of detectable CT signal upon addition of the non-infected patient samples (and the corresponding RNP signal) indicates that only target CT DNA was detected. Similarly accurate results were obtained when both the Helix Elite™ CT/NG control swabs and the CT/NG control nucleic acid were used to produce a combined sample.

After confirming that the assay worked in a singleplex format, i.e., detecting one pathogen from a sample at a time, an experiment was performed to assess the ability of the presently disclosed methods to detect multiple sexually transmitted pathogens in a single sample using a multiplex format.

Similar samples to the singleplex assays were prepared using the CT/NG controls. The singleplex assays for CT and NG were performed for the new samples. The new samples were also used in assays that used both the CT and NG primers and probes in the same sample, to assess multiplex detection of multiple pathogens using a single sample.

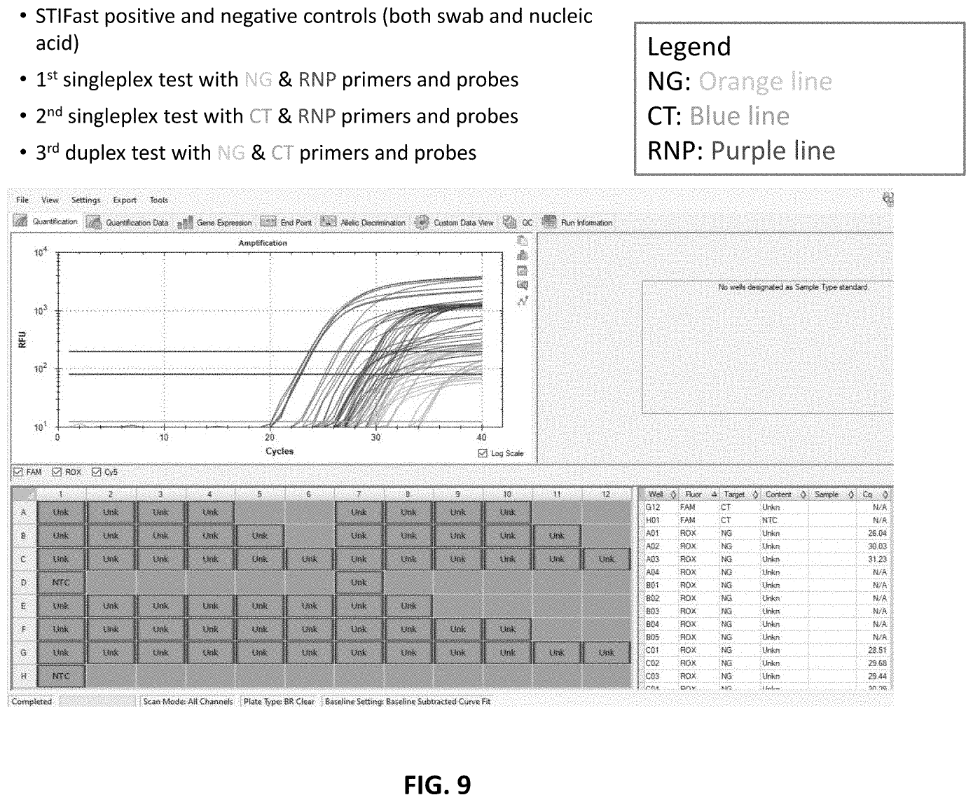

FIG. 9 provides the qPCR readouts from these assays.

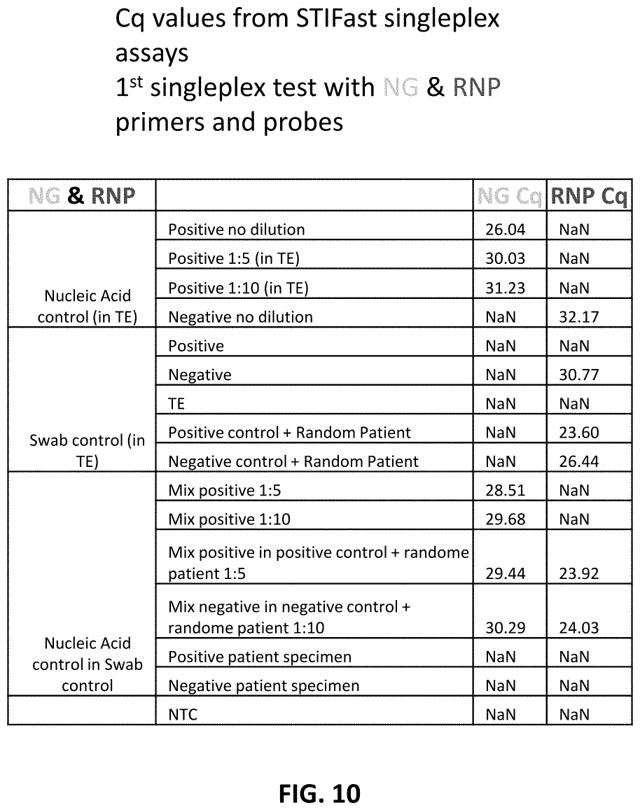

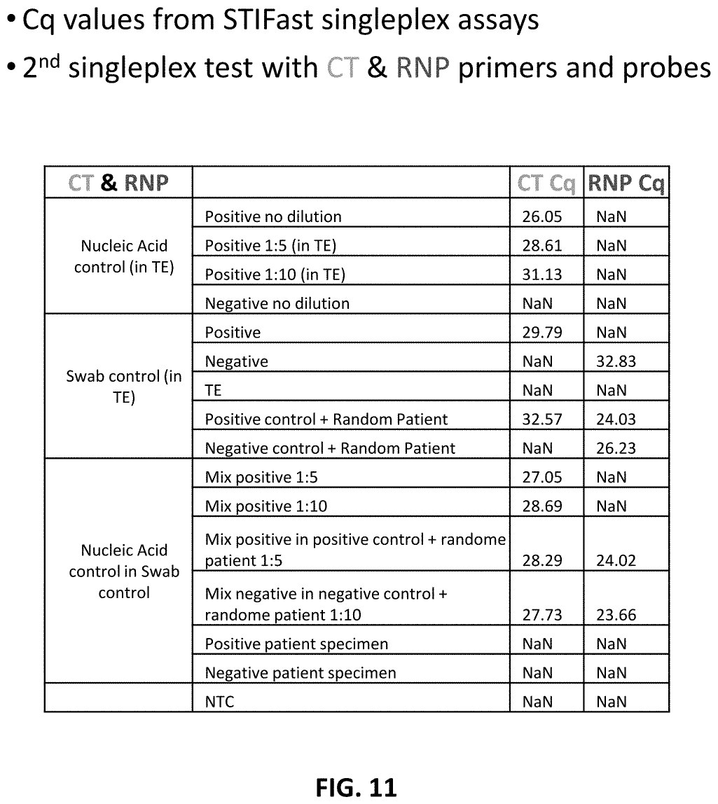

FIGS. 10-11 provide the qPCR Cq data and components used in the CT and NG singleplex assays. As shown, the data conforms with that obtained in the prior singleplex assays.

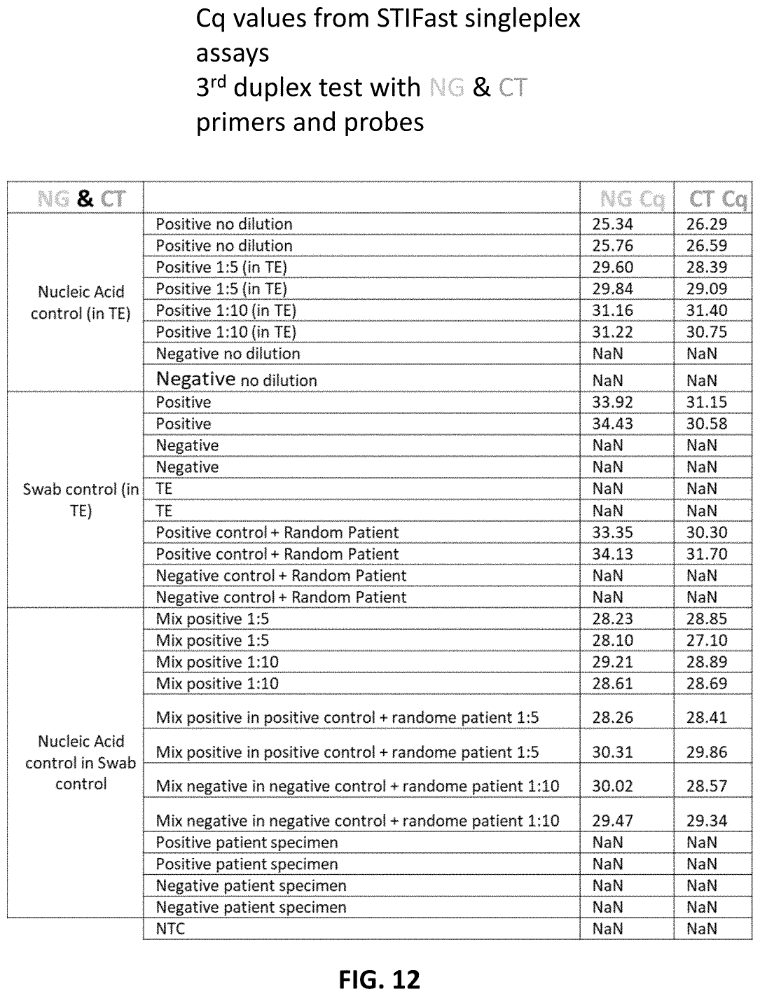

FIG. 12 provides the qPCR Cq data and components used in the CT and NG multiplex assays. As shown, both CT and NG were readily detected from the same samples by using both the CT and NG primers and probes.

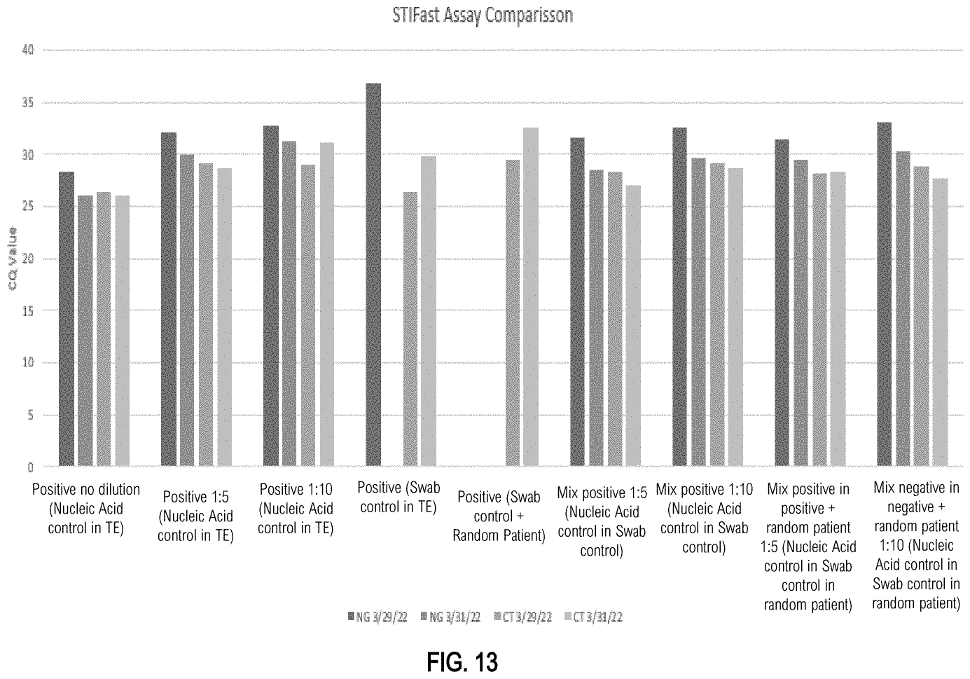

FIG. 13 provides a chart summarizing the results obtained in both the singleplex and multiplex assays. As shown, for both NG and CT detection, the results for each sample type were consistent across assays, which occurred on different days using new samples for each assay.

Accordingly, as shown, the methods of the invention are able to detect sexually transmitted pathogens from both swab and bodily fluid samples. Furthermore, the methods of the invention are able to detect a plurality of different sexually transmitted pathogens using a single sample. Moreover, the methods of the invention.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for extraction-free analysis of nucleic acid, the method comprising the steps of:
obtaining a sample comprising a mucosal membrane swab sample and/or a bodily fluid sample from a subject;
contacting the sample with a buffer composition comprising nuclease-free water, an antifungal, an antibiotic, and a ribonuclease inhibitor;
directly amplifying nucleic acid from the sample in said buffer with primers specific to one or more target nucleic acids of one or more sexually transmitted pathogens without prior extraction of said nucleic acid; and
analyzing amplicons produced in said amplifying step to detect one or more sexually transmitted pathogens in the subject.

2. The method of claim 1, wherein said mucosal membrane swab sample comprises one or more of a vaginal swab, a cervical swab, a urethral swab, a genital swab, a buccal swab, a throat swab, a nasal swab, ocular swab, and a combination of any thereof.

3. The method of claim 2, wherein said mucosal membrane swab sample comprises two or more of a vaginal swab, a cervical swab, a urethral swab, a genital swab, a buccal swab, a throat swab, a nasal swab, ocular swab, and combinations thereof.

4. The method of claim 2, wherein said sample comprises the mucosal membrane sample and a bodily fluid sample.

5. The method of claim 4, wherein said bodily fluid sample comprises one or more of mucous, blood, plasma, serum, serum derivatives, bile, maternal blood, phlegm, saliva, sputum, sweat, amniotic fluid, menstrual fluid, mammary fluid, follicular fluid of the ovary, fallopian tube fluid, peritoneal fluid, urine, semen, cerebrospinal fluid (CSF), or a combination thereof.

6. The method of claim 5, wherein said bodily fluid sample comprises urine.

7. The method of claim 5, wherein said bodily fluid sample comprises saliva.

8. The method of claim 1, wherein said sample comprises a bodily fluid sample comprising one or more of mucous, blood, plasma, serum, serum derivatives, bile, maternal blood, phlegm, saliva, sputum, sweat, amniotic fluid, menstrual fluid, mammary fluid, follicular fluid of the ovary, fallopian tube fluid, peritoneal fluid, urine, semen, cerebrospinal fluid (CSF), or a combination thereof.

9. The method of claim 1, wherein the one or more sexually transmitted pathogen is a virus and/or a bacterium.

10. The method of claim 9, wherein the one or more sexually transmitted pathogen comprises at least one of bacterial vaginosis, Chlamydia trachomatis (CT), cystitis, Neisseria gonorrhoeae (NG), hepatitis A, hepatitis B, hepatitis C, herpes (herpes simplex type 1 and 2), HIV, HPV, MPV, lymphogranuloma venereum, molluscum contagiosum, non-gonococcal urethritis, pelvic inflammatory disease, phthirus pubis, syphilis, trichomoniasis, and vaginitis.

11. The method of claim 10, wherein the target nucleic acids are from a plurality of sexually transmitted pathogens.

12. The method of claim 11, wherein the plurality of sexually transmitted pathogens comprise CT and/or NG.

13. The method of claim 12, wherein the nucleic acid specific primers comprise one or more of a primer having a sequence at least 75% identical to the nucleotide sequence set forth in any one of SEQ ID NOS: 1, 2, 4, and 5.

14. The method of claim 1, wherein prior to mixing the method comprises obtaining the bodily fluid sample in a vessel and placing a mucosal membrane swab into the bodily fluid sample in the vessel.

15. The method of claim 1, wherein said nucleic acid is RNA or DNA.

16. The method of claim 1, wherein said analyzing step comprises sequencing said amplicons.

17. The method of claim 1, wherein the buffer composition comprises a reducing agent.

18. The method of claim 17, wherein the reducing agent is a Tris(2-carboxyethyl)phosphine hydrochloride solution.

19. The method of claim 1, wherein the antifungal comprises an Amphotericin B and the antibiotic comprises Penicillin Streptomycin.

20. The method of claim 1, wherein the buffer composition stabilizes the nucleic acids from the sample.

21. The method of claim 1, further comprising the step of comparing target nucleic acid quantities in a plurality of samples obtained from the subject at successive time points and determining disease progression based on increases or decreases in the nucleic acid quantities over time.

22. The method of claim 1, wherein the wherein the amplicons are derived from an oncogenic virus.

23. The method of claim 1, wherein the target nucleic acid is an oncogene or portion thereof.
